(19) United States
(12) Patent Application Publication
(10) Pub. No.: US 2026/0241077 A1
(43) Pub. Date: Aug. 20, 2026

(54) Implantable Device, in Particular for the Reconstruction of the Hypodermis

(71) Applicant: Lattice Medical, Loos (FR)

(72) Inventors: Julien PAYEN, Fournes en Weppes (FR); Megann LE REST, Mons en Baroeul (FR); Shengheng CAO, Lille (FR); Damien CLERET, Loos (FR); Jaime DESTOUESSE, Lesquin (FR)

(21) Appl. No.: 19/153,868

(22) PCT Filed: Feb. 6, 2025
(86) PCT No.: PCT/EP2024/052874

§ 371 Date: Aug. 5, 2025

(30) Foreign Application Priority Data
Feb. 6, 2023 (FR) FR2301099

(51) Int. Cl. A61L 27/58; A61L 27/18; A61L 27/36; A61L 27/54; A61L 27/56; C08L 67/04

(52) U.S. Cl. CPC A61L 27/58; A61L 27/18; A61L 27/3604; A61L 27/54; A61L 27/56; C08L 67/04; A61L 2430/04; A61L 2430/34

(57) ABSTRACT

The present invention relates to a bioresorbable implantable device comprising a base structure comprising at least one base layer comprising:
an assembly A comprising a layer of a plurality of elongate elements A1 which are oriented in a longitudinal direction LA1, and a layer of a plurality of elongate elements A2; an assembly B comprising a layer of a plurality of elongate elements B1, oriented in a longitudinal direction LB1 and a layer of a plurality of elongate elements B2;
the assembly B is placed at least partially on top of the assembly A;
the direction LA1 intersects the direction LB1;
and the base structure comprises a plurality of base layers placed at least partially one on top of the other.

### TECHNICAL FIELD

The present invention relates to an implantable device, in particular for the reconstruction of the hypodermis, as well as a method for manufacturing said device. The present invention relates to an implantable device for replacing and/or reconstructing and/or increasing a volume of soft tissue, in particular after the removal of a volume of soft tissue from a subject, in particular a volume of adipose tissue.

Without limitation, the volume of soft tissue removed from the subject may be a tumor, a portion of hypodermis, a portion of adipose tissue or else a volume of breast tissue or a combination thereof.

### STATE OF THE ART

The skin is an envelope that covers our body. It represents ⅓ of the weight of the human body and has a surface area of approximately 2 m² in an adult.

The skin is a complex organ, divided into three overlapping layers that run from surface to depth: the epidermis, the dermis, and the hypodermis. The entire skin is separated from the underlying organs by fascia.

The epidermis, or superficial layer, protects the body against external aggressions. After the migration of keratinocytes, it forms a covering epithelium, which is stratified, squamous and keratinized.

Its thickness varies depending on the location and is determined by the thickness of the stratum corneum.

The epidermis is constantly renewing itself, taking an average of 21 days to renew itself. The dermis is a support and nutrition tissue for the epidermis. The dermis is a complex connective tissue, unlike the epidermis, it is richly vascularized. Its thickness is greater than that of the epidermis (0.5 to 2 mm compared to 0.1 mm). The dermis gives elasticity and tone to the skin due to the presence of fibrous proteins. The hypodermis or subcutaneous tissue separates the dermis from the underlying muscles and bones. The hypodermis is the innermost and thickest layer of the skin. The hypodermis has mechanical, aesthetic, protective and metabolic functions. It is formed of loose connective tissue containing fat cells. Its thickness varies greatly depending on the individual, age, body location and nutrition.

Many injuries can affect, and therefore destroy or damage, the hypodermis in more or less extensive areas of the body, with serious after-effects.

Every day, around the world, approximately 32000 serious injuries with serious after-effects in which the hypodermis is affected are observed. Said injuries include 3^{rd} and 4^{th} degree burns of the skin, tumor resections, or after frostbite, ulcers, or in the case of congenital defects. These injuries can be caused by explosive devices, firearms, or occur following transport accidents, or during domestic accidents.

A first approach to reconstructing the hypodermis involves transplanting an autologous flap, that is to say, a total, vascularized skin graft. This surgical technique is lengthy, difficult to perform, painful for the patient, and is only suitable for small wounds.

A second reconstruction approach involves intradermal injections of non-autologous filler (for example, hyaluronic acid). This type of surgery is minimally invasive, but the injected product is not autologous. Furthermore, the filler is not permanent. Finally, this type of reconstruction can only be performed on very small wounds.

A third way of reconstruction involves taking non-vascularized adipose tissue, treating it, then re-injecting it by transfer (for example, lipofilling) into the area to be treated. The advantage is that this type of surgery is minimally invasive but requires multiple interventions spaced out over time to achieve the desired appearance and/or to fill the loss of injected adipose tissue.

The removal and subsequent injection of adipose tissue with a cannula having a small internal diameter (usually around 4 mm) exerts shear and abnormal pressure on the cells, which reduces initial cell viability. In addition, damaged cells are reinjected into a restricted space without direct access to nutrients and growth factors via the vascular network. As a result, there is an unpredictable loss of volume of adipocyte tissue of up to 80%.

There is currently no effective, rapid, simple and affordable surgical solution for reconstructing soft tissue, particularly the hypodermis.

The absence of reconstruction causes psychological, aesthetic and functional after-effects in the patient.

There is therefore a need for an implantable device for reconstructing soft tissue that must remain flexible, compressible, lightweight, and cuttable into a specific shape while maintaining its structure, cohesion, and mechanical properties.

There is also a need for a bioresorbable implantable device configured to receive a given volume of initial adipose tissue before implantation and capable of retaining this volume of adipose tissue as much as possible without significant loss of volume or requiring the addition of a new volume of fat into the implantable device after implantation.

There is a need for an implantable device that improves the behavior of the implanted adipose tissue volume in terms of: cell migration and proliferation, adipose cell insertion and retention, and cell viability.

Finally, there is a need for an implantable device for reconstruction and/or replacement and/or augmentation of soft tissue that facilitates the surgical procedure, in particular by avoiding having to rework the cut edges of the implantable device after its shaping and filling it with a new volume of adipose tissue.

### DISCLOSURE OF THE INVENTION

The present invention relates, according to a first aspect, to an implantable device, in particular a bioresorbable device, in particular for the replacement and/or reconstruction and/or augmentation of soft tissue, for example for the reconstruction and/or replacement of the hypodermis or for the replacement and/or reconstruction and/or augmentation of breasts, comprising a base structure, said base structure comprising at least one base layer, and said base layer comprising:
an assembly A comprising a layer of a plurality of elongate elements A1, spaced apart from each other, and oriented in a longitudinal direction LA1, and a layer of a plurality of elongate elements A2, spaced apart from each other, and oriented in a longitudinal direction LA2, the directions LA1 and LA2 being intersecting;
an assembly B comprising a layer of a plurality of elongate elements B1, spaced apart from each other, and oriented in a longitudinal direction LB1 and a layer of a plurality of elongate elements B2, spaced apart from each other, and oriented in a longitudinal direction LB2, the directions LB1 and LB2 being intersecting;
the assembly B is placed at least partially on top, in particular is placed on top, of the assembly A;
the direction LA1 intersects the direction LB1;
and the implantable device comprises a base structure comprising a plurality of base layers placed at least partially one on top of the other.

The inventors have discovered that the implantable device according to the invention offers numerous advantages, in particular concerning the volume of adipose tissue removed by liposuction and introduced into the implantable device: improves cell migration and proliferation, facilitates insertion into the entire thickness of the base structure and the retention of fat cells in said structure, improves initial cell viability, and reduces long-term cell death.

Placing elongate elements selected within the framework of the present invention one on top of the other allows to create open and connected pores of various sizes allowing the reception and maintenance of different types of fats (for example macrofat, milifat, microfat or even nanofat, the name of which depends on the size of the fat collection cannula).

The implantable device also advantageously facilitates the surgical procedure because it is not necessary to re-inject a new volume of adipose tissue after implantation in the implantable device to fill the initial loss of adipose tissue.

Additionally, the adipose tissue taken can be spread to penetrate the thickness of the implantable device, which avoids shearing the cells when introducing them into the implantable device and thus improves their initial cell viability.

The implantable device is also advantageously divisible without loss of its cohesion, and can therefore be adapted by cutting to the dimensions of the anatomical area to be treated, in particular to be filled and/or reconstructed. The implantable device according to the invention is advantageously customizable while being able to be manufactured industrially.

The observed advantages were evaluated compared to a reconstruction technique using only a layer of adipose tissue placed in the defect to be treated.

The implantable device according to the invention is generally used for filling any anatomical defect comprising adipose tissue, in particular non-vascularized or vascularized, preferably non-vascularized.

The implantable device is preferably suitable for hypodermal reconstruction or breast reconstruction.

Advantageously, the implantable device is configured to allow the reconstruction and/or replacement of the hypodermis but is also compatible with techniques for reconstructing the dermis and the epidermis.

In this text, the base structure comprises n+1 base layers, with n being an integer greater than or equal to 1.

In this text, a base layer n+1 is similar in construction to a base layer n.

Preferably, a base layer n+1 is placed on top of a base layer n so that at least one elongate element A1 (or respectively A2) of the assembly A of the base layer n+1 is substantially parallel to at least one elongate element A1 (or respectively A2) of the assembly A of the base layer n.

Preferably, a base layer n+1 is placed on top of a base layer n without rotation of the assembly A of the layer n+1 relative to the assembly A of the layer n.

Preferably, a base layer n+1 is placed on top of a base layer n so that at least one elongate element A1 (in particular B1 or C1), and at least one elongate element A2 (in particular B2 or C2), of an assembly A (in particular of an assembly B or C) of the layer n+1 are substantially parallel respectively to at least one elongate element A1 (in particular B1 or C1), and at least one elongate element A2 (in particular B2 or C2), of an assembly A (in particular of the assembly B or C) of the layer n.

Preferably, a base layer n+1 is placed on top of a base layer n so that the direction LA1 (in particular LB1 or LC1) and the direction LA2 (in particular LB2 or LC2) of the layer n+1 are substantially parallel respectively to the direction LA1 (in particular LB1 or LC1) and to the direction LA2 (in particular LB2 or LC2), of an assembly A (in particular of an assembly B or C), of the layer n.

### IMPLANTABLE DEVICE

Advantageously, the base structure comprises open and connected pores.

The fat taken and applied to one side of the base structure thus diffuses through the base structure, and remains maintained in the base structure thanks to the array of open pores, connected to each other and having varied sizes.

In particular, the base structure comprises open pores having a size greater than 1 mm, open pores having a size greater than or equal to 1 μm and less than or equal to 1 mm, and open pores having a size less than or equal to 1 μm and greater than or equal to 1 nm.

In this text, open and connected pores are understood to mean that said pores are in fluid communication with each other.

Advantageously, the base structure, in particular the implantable device, is a plate having a determined thickness, in particular the general shape of which depends on the final application.

Advantageously, the base structure, in particular the implantable device, may be manufactured in the form of a rectangular or square plate or in any determined three-dimensional shape, for example in the shape of a dome, from which the structure may be resized, in particular by cutting, to conform to the defect of the subject to be treated.

In one embodiment, the base structure is manufactured, in particular in three dimensions, according to a tailor-made shape corresponding specifically to the patient's defect (the determined shape of the base structure is said to be patient-specific).

Preferably, the base structure is manufactured by three-dimensional printing.

The base structure, in particular the implantable device, can thus have any shape: rectangular, square, disc, or even dome.

Preferably, the base structure, in particular the implantable device, comprises a first face and a second face substantially opposite said first face. The first face is advantageously in fluid communication with the second face via open and connected pores.

Preferably, the base structure, in particular the implantable device, comprises open, connected pores opening onto its first face and onto its second face.

In one embodiment, the base structure, in particular the implantable device, has a substantially homogeneous determined thickness, in particular corresponding to the distance separating the first face from the second face of the base structure.

For example, the base structure, in particular the implantable device, has a substantially constant thickness comprised between 0.5 mm and 50 mm, preferably a thickness comprised between 1 mm and 20 mm, in particular for the reconstruction and/or replacement of the hypodermis.

For example, the base structure (in particular for the reconstruction and/or replacement of the hypodermis), in particular the implantable device, has a thickness greater than or equal to 2 mm or 3 mm or 4 mm or 4.5 mm, and preferably less than or equal to 15 mm or 10 mm or 8 mm or 6 mm or 5 mm.

In one embodiment, the base structure, in particular the implantable device, has a variable thickness, in particular corresponding to the distance separating the first face from the second face of the implantable device. For example, the base structure, in particular the implantable device, has a general dome shape.

For example, the base structure, in particular the implantable device, has a variable thickness comprised between 0.5 mm and 150 mm, preferably between 0.5 mm and 65 mm, in particular for breast reconstruction and/or replacement and/or augmentation.

In particular, when the base structure is a dome, the value of 65 mm corresponds to the top of the dome.

In this text, a value is comprised between a and b means that the lower bounds a and upper bounds b are comprised in said interval.

In one example, the implantable device comprises, in particular is essentially made up of, a plurality of base layers placed one on top of the other, and optionally an assembly A and/or an assembly B and/or an assembly C, and optionally one or more functional agent(s).

In one embodiment, in particular for the reconstruction and/or replacement of the hypodermis, the base structure comprises from 2 to 20 base layers, in particular from 3 to 20 base layers, preferably from 2 to 10 base layers, more preferably from 2 to 6 base layers, in particular from 3 to 5 base layers, in particular 3 base layers (in particular each comprising assemblies A, B and C) and a partial base layer consisting essentially of assemblies A and/or B.

In one embodiment, the base structure comprises 4 base layers, in particular each comprising assemblies A, B and C, and optionally at least one assembly A and/or B and/or C.

In one embodiment, a base layer (particularly comprising assemblies A, B and C) has a thickness comprised between 0.8 mm and 1.5 mm, preferably of the order of 1.1 mm to +/−0.1 mm.

In one embodiment, a base layer has a thickness comprised between 0.5 mm and 1.5 mm.

In one example, the base structure comprises, in particular is essentially made up of, a plurality of base layers placed one on top of the other, and optionally an additional assembly A and/or assembly B and/or assembly C.

The base layers are placed one on top of the other until the desired final thickness for the base structure, particularly for the implantable device, is obtained. It is possible for the last base layer to be only partial, that is to say it may only comprise one assembly A, or one assembly A and one assembly B, in order to achieve the desired final thickness precisely.

Advantageously, the volume of adipose tissue placed in the porous volume of the base structure, in particular the implantable device, is autologous.

Advantageously, the volume of adipose tissue placed in the porous volume of the base structure, in particular the implantable device, is not vascularized.

Advantageously, the implantable device, which is in particular bioresorbable, and/or the base structure, and/or at least one of the base layers and/or the base layers, comprises/comprise, in particular is/are essentially constituted of one or more (co)polymer(s), which is/are in particular bioresorbable, as described in this text.

### Functional Agent(s)

Advantageously, the base structure can be treated, for example impregnated or coated with one or more functional agent(s).

Preferably, a functional agent is any active substance, aiming at:
(i) improving cellular and tissue regeneration (for example promoting adipocyte growth, proliferation and/or differentiation) and/or maintaining the extracellular matrix; and/or
(ii) limiting or inhibiting the growth of microorganisms; and/or
(iii) inhibiting a localized inflammatory reaction and preventing the development of fibrosis.

Preferably, one or more functional agent(s):
is/are selected from a list I comprising (in particular consisting essentially of) growth factors promoting angiogenesis (pro-angiogenic), such as vascular endothelial growth factor (VEGF), platelet-derived growth factor (PDGF), stromal cell-derived factor 1 (SDF-1), any cellular fraction such as platelet-rich plasma (PRP) or platelet lysates; factors promoting cell proliferation, cell migration and adipocyte differentiation such as fibroblast growth factor (FGF), insulin-like growth factor (IGF), corticosteroids such as dexamethasone, hormones such as insulin, therapeutic agents such as 3-isobutyl-1 methylxanthine (IBMX); stromal vascular fraction (SVF); cell adhesion promoting factors such as fibronectin, polydopamine; and/or
is/are selected from a list II comprising (in particular consisting essentially of): cells selected from adipocytes, cells capable of differentiating into adipocytes, mixtures of cells selected from adipocytes and cells capable of differentiating into adipocytes, preferably said cells come from the patient being treated, or a mixture of the latter; and/or
is/are selected from a list III comprising (in particular consisting essentially of): anti-thrombotics, in particular anti-coagulants, such as heparin, anti-platelet agents such as aspirin, thrombolytics for destroying a blood clot in place; anti-parasitic molecules; anti-inflammatoires; vitamins; antiseptics (for example betadine); antibiotics; or a mixture of the latter; and/or
form(s) a bioresorbable polymer matrix, in particular the bioresorbable polymer matrix is a hydrogel, preferably the functional agent(s) is/are then selected from a list IV comprising (in particular consisting essentially of): cyclodextrins, cyclodextrin derivatives, gelatin, chitosans, lactic acid (co)polymer(s) (L or D form), glycolic acid (co)polymer(s), lactic acid and glycolic acid copolymers, lactic acid (L or D form) or glycolic acid and caprolactone copolymers, caprolactones, poly-p-dioxanone polymer, carrageenans, collagen or other molecules mimicking the structure and organization of the extracellular matrix, or a mixture thereof.

In one embodiment, the base structure is coated at least in part with functional agent(s) forming a bioresorbable polymer matrix, in particular selected from the list IV, optionally in admixture with one or more functional agent(s) selected from the list I and/or from the list II and/or from the list III.

Preferably, the functional agent(s) selected from the list II is/are one or more volumes of adipose tissue arranged at least partly in the porous volume of the base structure. Preferably, the volume(s) of adipose tissue is/are autologous.

First, the implantable device comprises a coating, in particular in a bioresorbable polymer matrix as described in this text, optionally said coating comprising one or more functional agent(s) selected from the list I and/or the list II and/or from the list III, fully or partially coating at least one face of the base structure, in particular fully or partially coating the first face and/or the second face of the base structure.

In a second example, optionally combined with the first example, at least a portion of the walls of the open and connected pores is coated with a coating, in particular in a bioresorbable polymer matrix as described in this text, optionally said coating comprising one or more functional agent(s) selected from the list I and/or the list II and/or from the list III, in particular said coating comprises at least one volume of adipose tissue.

### Elongate Element(s) A1 and/or A2 and/or B1 and/or B2 and/or C1 and/or C2 (Described in the Rest of the Text)

Preferably, an elongate element is a portion of a monofilament yarn or cord, in particular extruded from a mixture comprising at least one (co)polymer.

Thus, advantageously at least a portion of the elongate elements selected from the elongate elements A1 (in particular 1A1, 2A1), A2 (in particular 2A1, 1A2), B1 (in particular 1B1, 2B1), and B2 (in particular 1B2, 2B2), in particular C1 (in particular 1C1, 2C1) and C2 (in particular 1C2, 2C2), are portions of monofilament yarn(s) or cords, in particular extruded from a mixture comprising at least one (co)polymer, in particular the (co)polymer described in this text.

Preferably, the base structure is obtained by additive manufacturing, more particularly by three-dimensional printing of elongate elements A1, B1 and C1, and in particular A2, B2 and C2.

Preferably, the base structure comprises elongate elements A1 and/or A2 and/or B1 and/or B2 and/or C1 and/or C2 each comprising a portion having a diameter or a width comprised between 0.01 mm and 0.80 mm, more preferably between 0.01 mm and 0.50 mm, preferably between 0.01 mm and 0.30 mm, in particular between 0.10 mm and 0.30 mm.

Preferably, said elongate element, in particular said portion of monofilament yarn or said cord, comprises, in particular consists essentially of, at least one bioresorbable (co)polymer.

In one embodiment, at least a portion of the elongate elements selected from the elongate elements A1 (in particular 1A1, 2A1), A2 (in particular 2A1, 1A2), B1 (in particular 1B1, 2B1), and B2 (in particular 1B2, 2B2), in particular C1 (in particular 1C1, 2C1) and C2 (in particular 1C2, 2C2), comprises, in particular consists essentially of, at least one bioresorbable (co)polymer.

The (co)polymer can be a homopolymer or a copolymer or a terpolymer.

Said (co)polymer may be selected from: lactic acid (co)polymer(s), such as PLLA or PDLA, and PLA; ε-caprolactone (co)polymer(s); polydioxanones; glycolic acid (co)polymer(s), such as PLGA; glycolic acid and lactic acid (co)polymer(s); lactic acid and ε-caprolactone (co)polymer(s).

Preferably, said (co)polymer comprises at least two different repeating units.

Preferably, said (co)polymer is a ε-caprolactone polymer and at least one repeating unit different from ε-caprolactone, for example derived from lactic acid and/or derived from glycolic acid.

Preferably, the repeating units derived from lactic acid may be L-form lactic acid repeating units and/or D-form lactic acid repeating units and/or D, L-form lactic acid repeating units.

Preferably, said at least one (co)polymer comprises repeating units of ε-caprolactone whose molar fraction (in said (co)polymer) is less than or equal to 50%, more preferably less than or equal to 40%, in particular between 20% and 40% (limits included).

Preferably, said at least one (co)polymer comprises repeating units of L-form lactic acid and/or repeating units of D-form lactic acid and/or D, L-form lactic acid, the molar fraction of which (in said (co)polymer) is greater than or equal to 50%, more preferably greater than or equal to 60%, in particular between 60% and 80% (limits included).

Said molar fractions can be determined by NMR spectroscopy.

Preferably, said at least one (co)polymer comprises repeating units of L-form lactic acid, and/or repeating units of D-form lactic acid and/or D, L-form lactic acid, the molar fraction of which is less than or equal to 90%, more preferably less than or equal to 80%.

In this text, an element consists essentially of one or more sub-element(s) and is understood to mean that said sub-element(s) represent(s) at least 80% by mass or volume, in particular at least 90% by mass or volume, of said sub-element(s).

Advantageously, the layers of elongate elements A1 and A2 are placed one on top of the other.

Advantageously, the layer of elongate elements A2 is placed above the layer of elongate elements A1.

Advantageously, the elongate elements A1 are arranged substantially parallel to each other, and spaced apart from each other.

Advantageously, the elongate elements A2 are arranged substantially parallel to each other, and spaced apart from each other.

Advantageously, the layers of elongate elements B1 and B2 are placed one on top of the other.

Advantageously, the layer of elongate elements B2 is placed above the layer of elongate elements B1.

Advantageously, the elongate elements B1 are arranged substantially parallel to each other, and spaced apart from each other.

Advantageously, the elongate elements B2 are arranged substantially parallel to each other, and spaced apart from each other.

Advantageously, the assembly B is placed on top of the assembly A according to an angle of rotation corresponding to the angle formed at the intersection of the directions LA1 and LB1.

The angle of rotation is measured clockwise, and therefore inversely to the trigonometric direction.

This arrangement allows the generation of open and connected pores of various sizes and shapes.

In an alternative embodiment, the base layer comprises an assembly C comprising a layer of a plurality of elongate elements C1, spaced apart from each other, and oriented in a longitudinal direction LC1 and a layer of a plurality of elongate elements C2, spaced apart from each other, and oriented in a longitudinal direction LC2, the directions LC1 and LC2 being intersecting, the assembly C is placed at least partially on top of the assembly B, in particular the direction LC1 intersects the direction LB1. It has also been advantageously observed that blood vessels can be established from the assembly A to the assembly C in a base layer via the interconnected pores. Thus, vascularization of the base structure is observed throughout its thickness.

Advantageously, placing three assemblies A, B and C one on top of the other allows to create an array of open and connected pores having even more varied sizes and facilitating the reconstruction of the soft tissue.

Advantageously, the layers of elongate elements C1 and C2 are placed one on top of the other.

Advantageously, the layer of elongate elements placed at least partially on top of the C2 is placed above the layer of elongate elements C1.

Advantageously, the elongate elements C1 are arranged substantially parallel to each other, and spaced apart from each other.

Advantageously, the elongate elements C2 are arranged substantially parallel to each other, and spaced apart from each other.

Advantageously, the assembly C is placed on top of the assembly B according to an angle of rotation corresponding to the angle formed at the intersection of directions LB1 and LC1.

In an alternative embodiment, the direction LA1 intersects the direction LB1 and forms at their intersection an angle λ1 greater than or equal to 10° and less than or equal to 60°, in particular greater than or equal to 20° and less than or equal to 45°, in particular greater than or equal to 30° and less than or equal to 40°, for example of the order of 36°.

In an alternative embodiment, the assembly B is placed on top of the assembly A while being pivoted by said angle λ1.

Advantageously, the elongate elements B1 are pivoted by said angle λ1 relative to the elongate elements A1.

In an alternative embodiment, the direction LA1 intersects the direction LC1 and forms at their intersection an angle λ3 greater than λ1 by at least 10°, in particular approximately twice λ1. Preferably, λ3 is greater than or equal to 40° and less than or equal to 90°, more preferably greater than or equal to 60° and less than or equal to 80°.

Advantageously, the assembly C is placed on top of the assembly B while being pivoted by an angle λ2 of at least 10°. Preferably, the angle λ2 is greater than or equal to 10° and less than or equal to 60°, in particular greater than or equal to 20° and less than or equal to 45°, in particular greater than or equal to 30° and less than or equal to 40°, for example of the order of 36°. Preferably, the angle λ2 is substantially equal to λ1.

Advantageously, the assemblies A, B and C are not placed one on top of the other in a base layer so that the elongate elements A1, B1 and C1 are aligned in parallel directions.

The placement of the assemblies A, B and C one on top of the other in this way allows to secure the layers of elongate elements (A1, A2, B1, B2, C1, C2) together and also allows to increase the contact surface between these layers. If such a rotation were not carried out, conduits with a perfectly reproducible shape would be obtained.

It has also been observed that this arrangement advantageously contributes to the placement and orientation of blood vessels in the assembly A up to the assembly C in a base layer via the interconnected pores. Thus a vascularization of the base structure throughout its thickness is advantageously observed.

In an alternative embodiment, the angle formed between the direction LA1 and the direction LA2 is greater than or equal to 70° and less than or equal to 110°, in particular comprised between 80° and 100°, more particularly comprised between 85° and 95°, in particular approximately 90°.

In an alternative embodiment, the angle formed between the direction LB1 and the direction LB2 is greater than or equal to 70° and less than or equal to 110°, in particular comprised between 80° and 100°, more particularly between 85° and 95°, in particular approximately 90°.

In an alternative embodiment, the angle formed between the direction LC1 and the direction LC2 is greater than or equal to 70° and less than or equal to 110°, in particular comprised between 80° and 100°, more particularly comprised between 85° and 95°, in particular approximately 90°.

In an alternative embodiment, the distance separating two adjacent elongate elements A1, respectively A2, in a layer of a plurality of elongate elements A1, respectively A2, of an assembly A is comprised between 2 mm and 8 mm, preferably comprised between 3 mm and 6 mm, more preferably comprised between 3.5 mm and 4.5 mm, in particular of the order of 4 mm.

This arrangement allows to play on the size of the open pores, and to offer a compromise in order to retain small cellular aggregates (for example microfat) and larger cellular aggregates (for example macrofat).

It has also been observed that this distance promotes good penetration, dispersion and retention of fatty tissue throughout the thickness of the base structure.

In an alternative embodiment, the distance separating two adjacent elongate elements B1, respectively B2, in a layer of a plurality of elongate elements B1, respectively B2, of an assembly B is comprised between 2 mm and 8 mm, preferably comprised between 3 mm and 6 mm, more preferably comprised between 3.5 mm and 4.5 mm, in particular of the order of 4 mm.

In an alternative embodiment, the distance separating two adjacent elongate elements C1, respectively C2, in a layer of a plurality of elongate elements C1, respectively C2, of an assembly C is comprised between 2 mm and 8 mm, preferably comprised between 3 mm and 6 mm, more preferably comprised between 3.5 mm and 4.5 mm, in particular of the order of 4 mm.

In an alternative embodiment, at least one elongate element A1 is secured to several elongate elements A2 by means of solidified thermofused portions originating from said elongate element A1 and/or the elongate elements A2.

The elongate elements A2 are placed one on top of the other, in a partially softened state, on the elongate elements A1, also in a partially softened state, so that they adhere to each other at their intersections, which creates anchor points between the layer of elements A1 and the layer of elements A2.

This arrangement promotes the cohesion and tear resistance of the base structure, and thus allows it to be cut to the desired dimensions without loss of its structure.

In an alternative embodiment, at least one elongate element B1 is secured to several elongate elements B2 by means of solidified thermofused portions originating from said elongate element B1 and/or the elongate elements B2.

In an alternative embodiment, at least one elongate element C1 is secured to several elongate elements C2 by means of solidified thermofused portions originating from said elongate element C1 and/or the elongate elements C2.

In an alternative embodiment, at least one elongate element A1 comprises at least one portion a11 of its length in a zigzag or sinusoidal shape.

Advantageously, said portion a11 comprises troughs and peaks.

This arrangement allows to improve the flexibility, and therefore the ability to deform, of the assembly A, and therefore of the base layer, and ultimately of the base structure.

Advantageously, at least one elongate element A2 comprises at least one portion a22 of its length in a zigzag or sinusoidal shape, in particular comprising troughs and peaks.

Advantageously, at least one elongate element B1 comprises at least one portion b11 of its length in a zigzag or sinusoidal shape, in particular comprising troughs and peaks.

Advantageously, at least one elongate element B2 comprises at least one portion b22 of its length in a zigzag or sinusoidal shape, in particular comprising troughs and peaks.

Advantageously, at least one elongate element C1 comprises at least one portion c11 of its length in a zigzag or sinusoidal shape, in particular comprising troughs and peaks.

Advantageously, at least one elongate element C2 comprises at least one portion C22 of its length in a zigzag or sinusoidal shape, in particular comprising troughs and peaks.

In an alternative embodiment, at least one elongate element A2 comprises at least one portion a22 of its length in a zigzag or sinusoidal shape, said portion a22 comprising troughs spaced apart from each other, and the portion a11 of said elongate element A1 is placed on top of the portion a22 of said elongate element A2 passing through a trough of said portion a22 in a zigzag or sinusoidal shape.

This arrangement allows the stacking of layers of elongate elements to be blocked there between, and provides stability to the base structure.

In the preceding variants, preferably a zigzag or sinusoidal portion is repeated over the entire length of an elongate element, in particular A1 and/or A2 and/or B1 and/or B2 and/or C1 and/or C2.

In one variant, at least one elongate element A1, respectively B1 or C1, comprises at least one sinusoidal or zigzag portion comprising troughs and peaks, and at least one elongate element A2, respectively B2 or C2, comprises at least one sinusoidal or zigzag portion comprising troughs and peaks, said at least one elongate element A1, respectively B1 or C1, is placed on top of said at least one elongate element A2, respectively B2 or C2, so that the troughs, respectively the peaks, of said at least one elongate element A1 cooperate with the peaks, respectively the troughs, of said at least one elongate element A2.

In an alternative embodiment, the base structure has a density greater than or equal to 0.01 g/cm³ and less than or equal to 1 g/cm³, preferably less than or equal to 0.1 g/cm³, more preferably less than or equal to 0.05 g/cm³. In one variant, at least a portion of the elongate elements selected from the elongate elements A1, A2, B1, and B2, in particular C1 and C2, are portions of monofilament yarns each having a diameter or a width less than or equal to 0.60 mm, preferably less than or equal to 0.40 mm, in particular comprised between 0.10 mm and 0.30 mm, in particular of the order of 0.20 mm.

In an alternative embodiment, the base structure comprises open and connected micropores, open and connected millipores, and open and connected macropores.

In this text, micropores are understood to mean pores having at least one dimension less than or equal to 1.2 mm, in particular between 0.1 mm and 1.2 mm.

The size of the micropores of the base structure is advantageously adapted to receive the cellular aggregates corresponding to the category called microfat category and taken with a cannula whose internal diameter is less than or equal to 1.2 mm and preferably greater than or equal to 0.1 mm.

In this text, millipores are understood to mean pores having at least one dimension less than or equal to 2.4 mm, in particular comprised between 1.2 mm and 2.4 mm. The size of the millipores of the base structure is advantageously adapted to receive the cellular aggregates corresponding to the category called millifat category and taken with a cannula whose internal diameter is less than or equal to 2.4 mm and preferably greater than or equal to 1.2 mm.

In this text, macropores are understood to mean pores having at least one dimension greater than 2.4 mm, in particular comprised between 2.4 mm and 10 mm.

The size of the macropores of the base structure is advantageously adapted to receive the cellular aggregates corresponding to the category called macrofat category and taken with a cannula whose internal diameter is greater than 2.4 mm, preferably greater than or equal to 4 mm, still preferably less than or equal to 10 mm.

It has thus been observed that the base structure advantageously comprises micropores, millipores and macropores whose sizes are adapted to receive different sizes of cellular aggregates in relation to the different internal diameters of the cannulas for collecting said adipose cells. In an alternative embodiment, at least 50% in number of the open pores of the base structure have at least one dimension less than or equal to 1.25 mm.

In an alternative embodiment, at least 10% in number of the open pores of the base structure have at least one dimension greater than or equal to 1.25 mm.

In an alternative embodiment, at least 10% in number of the open pores of the base structure have at least one dimension greater than or equal to 1.25 mm and less than or equal to 2.5 mm.

In an alternative embodiment, at least 5% in number of the open pores of the base structure have at least one dimension greater than or equal to 2.5 mm.

In an alternative embodiment, the base structure comprises from 50% to 65% in number of pores having a diameter less than or equal to 1.25 mm, in particular greater than or equal to 0.5 mm, from 25% to 45% in number of pores having a diameter comprised between 1.25 mm and 4 mm, and from 5% to 10% in number of pores having a diameter greater than or equal to 4 mm.

In one variant, at least 40% in number, preferably at least 60% in number, more preferably at least 80% in number, of the pores of the base structure have a size greater than or equal to 0.5 mm. Preferably, the method for determining the pore size and their number distribution (%), preferably comprises the following steps:
Image capture with a binocular using a x10 objective with an apparent ruler, particularly seen from above;
Measurement for each test of the size of approximately 100 pores on a sample, repeating the measurement at least 3 times;
Uploading in the ImageJ 1.53t software;
Definition of the image in 8-bit using the “Image>Type>8-bit” tool;
Definition of the scale using the “Analysis>Set Scale” tool using the visible ruler;
Binarization of the image with the “Image>Adjust>Threshold” tool;
Highlighting of pores by variation of limits;
“Analyze>Analyze particles” with Size(pixel^{∧}2): 2-Infinity, Circularity [0.00-1.00] and the “exclude on edges” box checked;
Saving the generated table in csv;
Uploading of the table into Excel by separating the information to obtain a column “” (count), “Area”, “Mean”;
Class distribution;
Determination of the percentage of each class.

Preferably, the size of the pores is measured by optical microscopy of sections (therefore in two dimensions) of the base structure or of the implantable device, in particular an average is taken over approximately 100 measurements taken along each of the three axes X, Y, and Z (in particular 3D printing of the base structure).

The pore size can be measured on the raw base structure or on a base structure comprising one or more functional agent(s).

In one variant, at least a portion of the elongate elements selected from the elongate elements A1, A2, B1, and B2, in particular C1 and C2, comprises, in particular is essentially made up of, at least one bioresorbable (co)polymer, preferably comprising repeating units of ε-caprolactone and repeating units of lactide of L or D or L, D form. Said copolymer can be written in the form poly(L-lactide or D-lactide or L, D-lactide-co-ε-caprolactone), in particular poly(L-lactide-co-ε-caprolactone).

In one variant, said bioresorbable (co)polymer has a number-average molar mass Mn greater than or equal to 10 000 g/mol, preferably greater than or equal to 25 000 g/mol, more preferably greater than or equal to 45 000 g/mol, in particular greater than or equal to 65 000 g/mol.

In one variant, said bioresorbable (co)polymer has a number-average molar mass Mn less than or equal to 200 000 g/mol, preferably less than or equal to 175 000 g/mol, more preferably less than or equal to 150 000 g/mol, in particular less than or equal to 120 000 g/mol or 100 000 g/mol.

In one variant, said bioresorbable (co)polymer has a mass-average molar mass Mw greater than or equal to 50 000 g/mol, preferably greater than or equal to 75 000 g/mol, more preferably greater than or equal to 100 000 g/mol, in particular greater than or equal to 125 000 g/mol.

In one variant, said bioresorbable (co)polymer has a number-average molar mass Mw of less than or equal to 300 000 g/mol, preferably less than or equal to 275 000 g/mol, more preferably less than or equal to 250 000 g/mol, in particular less than or equal to 225 000 g/mol or 200 000 g/mol.

In one variant, the polydispersity index of said at least one bioresorbable (co)polymer is less than or equal to 2.5 or 2.3 or 2.1.

In one variant, the polydispersity index (I=Mw/Mn) of the bioresorbable (co)polymer is greater than or equal to 1 or 1.3 or 1.5.

In one variant, at least a portion, or essentially all, of the elongate elements selected from the elongate elements A1, A2, B1, and B2, in particular C1 and C2, comprises at least one bioresorbable (co)polymer, said at least one (co)polymer comprises:
repeating units of ε-caprolactone whose molar fraction (in said (co)polymer) is less than or equal to 50%, more preferably less than or equal to 40%, in particular comprised between 20% and 40%, and/or
repeating units of lactic acid of L form and/or repeating units of lactic acid of D form and/or of D, L form whose molar fraction (in said (co)polymer) is greater than or equal to 50%, more preferably greater than or equal to 60%, in particular comprised between 60% and 80%.

In an alternative embodiment, at least a portion, or essentially all, of the elongate elements selected from the elongate elements A1, A2, B1, and B2, in particular C1 and C2, comprises, in particular is essentially made up of, at least one bioresorbable (co)polymer having an elongation at break (%) greater than or equal to 50%, preferably greater than or equal to 100%, more preferably greater than or equal to 150%, preferentially greater than or equal to 200%, in particular measured in the printing direction, X or Y, in three dimensions of the specimen tested.

In one embodiment, the bioresorbable (co)polymer has an elongation at break (%) less than or equal to 2000%, preferably less than or equal to 1500%, more preferably less than or equal to 1200%, in particular measured according to the printing direction, X or Y, in three dimensions of the specimen tested.

In one embodiment, the bioresorbable (co)polymer has a Young's modulus greater than or equal to 50 MPa, preferably greater than or equal to 75 MPa, in particular greater than or equal to 100 MPa (preferably regardless of the three-dimensional printing direction of the specimen tested: X, Y, Z).

In one embodiment, the bioresorbable (co)polymer has a Young's modulus less than or equal to 800 MPa, in particular less than or equal to 600 MPa (preferably regardless of the three-dimensional printing direction of the specimen tested: X, Y, Z).

Young's modulus and elongation at break are measured according to ASTM D638-14 (“Standard Test Method for Tensile Properties of Plastics”) at a tensile speed of 10 mm/min on type V specimens, preferably at a temperature of 20° C. The specimens tested are preferably manufactured by an additive manufacturing method, in particular the printing direction can vary along the axes X, Y or Z.

In one variant, the base structure has a porosity greater than or equal to 90%.

Preferably, the porosity is determined by the following calculation formula: (total volume of the base structure−void volume of the base structure)/total volume of the base structure*100.

Preferably, the total volume and the full volume of the base structure are estimated using the SOLIDWORKS 2019-SPO4 design software (the procedure used is: 1/“evaluate” tab, 2/Surface selection, 3/ “measurement” option, 4/surface value).

In an alternative embodiment, the implantable device, in particular the base structure, has a Young's modulus greater than or equal to 5 KPa, preferably less than or equal to 80 KPa, more preferably comprised between 5 KPa and 60 KPa, in particular comprised between 5 KPa and 50 KPa, for example comprised between 5 KPa and 25 KPa or else of the order of 10 KPa to +/−5 KPa.

Young's modulus is advantageously measured according to ASTM D1621-16 “Standard Test Method for Compressive Properties of Rigid Cellular Plastics”, in particular under the conditions described with reference to FIG. 13.

The present invention relates, according to a second aspect, to a method for manufacturing an implantable device, in particular according to any one of the variant embodiments with reference to the first aspect of the invention, said method comprises the steps:
a step of manufacturing a base layer (i), said step comprising:
a step (i1) of manufacturing an assembly A comprising the deposition of a plurality of elongate elements A1, in particular extruded, spaced apart from each other, and oriented in a longitudinal direction LA1, so as to form a layer, then the deposition of a plurality of elongate elements A2, in particular extruded, spaced apart from each other, and oriented in a longitudinal direction LA2, so as to form a layer arranged above the previous layer comprising the elongate elements A1, the directions LA1 and LA2 being intersecting; and
a step (i2) of manufacturing an assembly B comprising the deposition of a plurality of elongate elements B1, in particular extruded, spaced apart from each other, and oriented in a longitudinal direction LB1, so as to form a layer, then the deposition of a plurality of elongate elements B2, in particular extruded, spaced apart from each other, and oriented in a longitudinal direction LB2, so as to form a layer arranged above the previous layer comprising the elongate elements B1, the directions LB1 and LB2 being intersecting;
the assembly B is placed at least partially on top of the assembly A;
the direction LA1 intersects the direction LB1;
and the implantable device comprises a base structure comprising a plurality of base layers placed at least partially one on top of the other.

In one embodiment, said method comprises a step (i3) of manufacturing an assembly C comprising the deposition of a plurality of elongate elements C1, spaced apart from each other, and oriented in a longitudinal direction LC1, so as to form a layer, then the deposition of a plurality of elongate elements C2, spaced apart from each other, and oriented in a longitudinal direction LC2, so as to form a layer arranged above the previous layer comprising the elongate elements C1, the directions LC1 and LC2 being intersecting;
the assembly C is placed at least partially on top of the assembly B;
the direction LC1 intersects the direction LB1.

In one example, the implantable device comprises, in particular is essentially made up of, a plurality of base layers placed one on top of the other, and optionally an assembly A and/or an assembly B and/or an assembly C, and optionally one or more functional agent(s).

Advantageously, the method comprises several steps (i) carried out one after the other, optionally followed by carrying out a step (i1) or carrying out a step (i1) followed by a step (i2), until the desired thickness is obtained for the base structure.

In one variant, the implantable device comprises a number of base layers n+1 with n being an integer greater than or equal to 1.

In one variant, a base layer n+1 is placed on top of a base layer n so that at least one elongate element A1 (or respectively A2) of the assembly A of the base layer n+1 is substantially parallel to at least one elongate element A1 (or respectively A2) of the assembly A of the base layer n.

Preferably, a base layer n+1 is placed on top of a base layer n without rotation of the assembly A (or respectively B or C) of the layer n+1 relative to the assembly A (or respectively B or C) of the layer n.

Preferably, a base layer n+1 is placed on top of a base layer n so that at least one elongate element A1 (in particular B1 or C1), and at least one elongate element A2 (in particular B2 or C2), of an assembly A (in particular of an assembly B or C) of the layer n+1 are substantially parallel respectively to at least one elongate element A1 (in particular B1 or C1), and at least one elongate element A2 (in particular B2 or C2), of an assembly A (in particular of the assembly B or C) of the layer n.

Preferably, a base layer n+1 is placed on top of a base layer n so that the direction LA1 (in particular LB1 or LC1) and the direction LA2 (in particular LB2 or LC2) of the layer n+1 are substantially parallel respectively to the direction LA1 (in particular LB1 or LC1) and to the direction LA2 (in particular LB2 or LC2), of an assembly A (in particular of an assembly B or C), of the layer n.

In one variant, the base layer further comprises an assembly C comprising a layer of a plurality of elongate elements C1, spaced apart from each other, and oriented in a longitudinal direction LC1 and a layer of a plurality of elongate elements C2, spaced apart from each other, and oriented in a longitudinal direction LC2, the directions LC1 and LC2 being intersecting, the assembly C is placed at least partially on top of the assembly B, in particular the direction LC1 intersects the direction LB1.

The variants, embodiments according to a first aspect of the invention apply independently of each other to the second aspect of the invention.

The present invention also relates, according to a third aspect, to a method for ex-vivo manufacturing of an implantable device for the replacement and/or reconstruction and/or for increasing a volume of soft tissue comprising:
the provision of an implantable device according to any one of the variant embodiments with reference to the first aspect of the invention or obtained by the manufacturing method according to a second aspect of the invention, said base structure comprises a determined porous volume, and
the arrangement, at least in part in said porous volume of the base structure, of cells selected from adipocytes, cells capable of differentiating into adipocytes and mixtures of these two types of cells, said cells preferably originating from said subject.

Preferably, adipocytes are understood to mean all cells from the adipocyte lineage, from the mesenchymal stem cell to the mature adipocyte, including all intermediate stages such as pre-adipocytes for example.

In one embodiment, the fatty tissue, in particular cells selected from adipocytes, cells capable of differentiating into adipocytes and mixtures of these two types of cells, said cells preferably originating from said subject, is/are mixed with one or more functional agent(s) as described in this text, in particular with reference to the first aspect of the invention selected from the list I and/or the list III and/or the list IV.

This arrangement allows to improve the cellular response, in particular when the implantable device is intended for the treatment of a hypodermal defect.

The variant embodiments, as well as the definitions and embodiments according to a first aspect of the invention, can be combined with each other independently of each other, and with the variants according to a second aspect and/or a third aspect of the invention.

The present invention relates, according to a fourth aspect, to an implantable device obtained by implementing a manufacturing method as described with reference to the second aspect of the invention or to the third aspect of the invention.

### DESCRIPTION OF THE DRAWINGS

The invention will be better understood upon reading the following description of exemplary embodiments of the invention given as non-limiting examples, with reference to the appended drawings, in which:

FIG. 1 schematically shows, in top view, a first example of assembly A according to the invention;

FIG. 2 schematically shows, in side view, the first example of assembly A according to the invention;

FIG. 3 schematically shows a first example of placement of assemblies A, B and C one on top of the other for the formation of a first example of base layer according to the invention;

FIG. 4 schematically shows a second example of a base layer according to the invention;

FIG. 5 schematically shows an isometric view of the second example of base layer shown in FIG. 4;

FIG. 6 schematically shows a front view of the second example of base layer shown in FIGS. 4 and 5;

FIG. 7 schematically shows a side view of the second example of base layer shown in FIGS. 4, 5 and 6;

FIG. 8 is a photograph obtained by tomography, in top view, of a first example of an implantable device according to the invention;

FIG. 9 is a photograph obtained by tomography, in perspective view, of the first example of an implantable device shown in FIG. 8;

FIG. 10 is a top view photograph of an enlarged portion of the first example of an implantable device shown in FIGS. 8 and 9;

FIG. 11 is a photograph obtained by tomography, in perspective and top view, of the first example of an implantable device shown in FIGS. 8 to 10;

FIG. 12 is a graph showing the distribution (% in number) of pores according to their sizes in the base structure of the first example of implantable device shown in photographs 8 and 9;

FIG. 13 is a photograph of a histological section after two months of subcutaneous implantation in rats of the first example of implantable device shown in photographs 8 to 11;

FIG. 14 is a photograph of a portion of the first example of an implantable device showing angiogenesis using the model of the chicken Chorioallantoic Membrane;

FIG. 15 is a graph showing on the ordinate the Young's modulus (kPa) measured for a volume of adipose tissue and the Young's modulus (kPa) measured for the first example of an implantable device.

### DESCRIPTION OF THE EMBODIMENTS

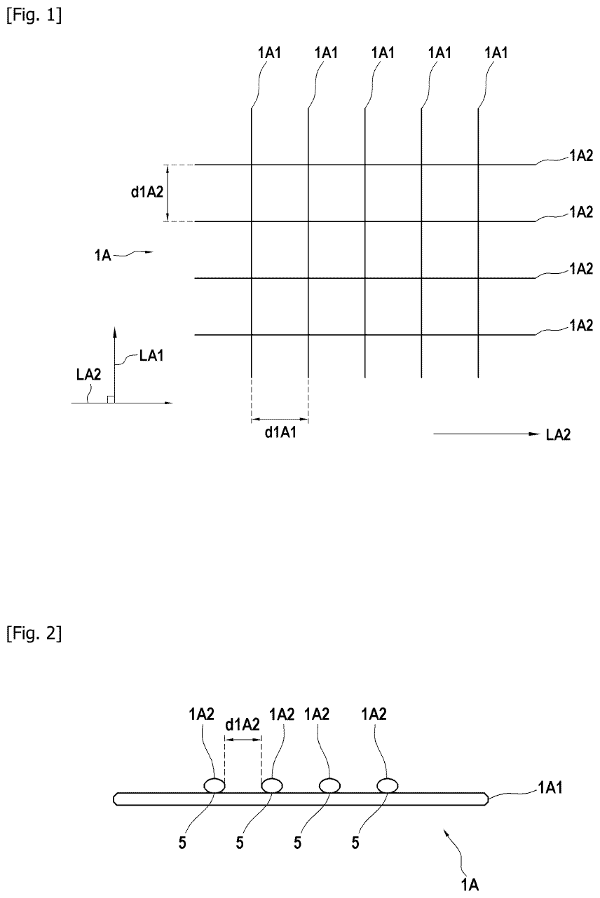

FIG. 1 schematically shows a first example of an assembly 1A comprising a layer of a plurality of elongate elements 1A1, spaced apart from each other by a distance d_{1A1}, and oriented in a longitudinal direction LA1, as well as a layer of a plurality of elongate elements 1A2, spaced apart from each other by a distance d_{1A2}, and oriented in a longitudinal direction LA2, the directions LA1 and LA2 being intersecting, in particular forming there between an angle comprised between 85° and 95°, in particular of approximately 90°. The elongate elements 1A1 are substantially parallel to each other. Similarly, the elongate elements 1A2 are substantially parallel to each other.

The elongate elements 1A2 are extruded and deposited by being placed one on top of the other in a layer on the layer of elongate elements 1A1. The elongate elements 1A2 are thus secured to the elongate elements 1A1 by means of thermofused polymer portions 5, shown in FIG. 2, from the elongate elements 1A2 and/or the elongate elements 1A1. When depositing the elongate elements 1A2, the elongate elements 1A1 were previously extruded and deposited in an aligned manner on a support, in particular a non-stick support.

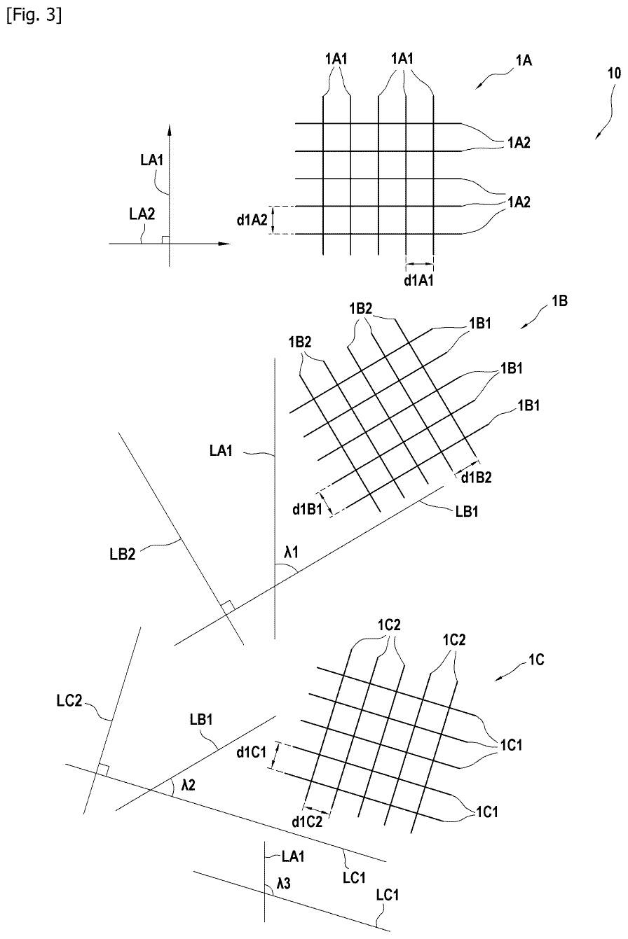

FIG. 3 schematically shows the placement of an assembly 1B on top of the assembly 1A shown in FIGS. 1 and 2, the assembly 1B being pivoted relative to the assembly 1A by an angle λ1 formed between the directions LA1 and LB1, in particular comprised between 30° and 40°, for example of the order of 36°. FIG. 3 also schematically shows the placement of an assembly 1C on top of the assembly 1B, the assembly 1C being pivoted relative to the assembly 1B by an angle λ2 formed between the directions LB1 and LC1, in particular comprised between 30° and 40°, for example of the order of 36°. The assemblies 1A, 1B and 1C thus placed one on top of the other form a base layer 10 according to the invention.

As shown in FIG. 3, the assembly 1B comprises a layer of a plurality of extruded elongate elements 1B1, spaced apart from each other by a distance d_{1B1}, and oriented in a longitudinal direction LB1 and a layer of a plurality of extruded elongate elements 1B2, spaced apart from each other by a distance d_{1B2}, and oriented in a longitudinal direction LB2, the directions LB1 and LB2 being intersecting, in particular forming there between an angle comprised between 85° and 95°, in particular of approximately 90°. The elongate elements 1B1 are substantially parallel to each other. Similarly, the elongate elements 1B2 are substantially parallel to each other.

Similarly, the assembly 1C comprises a layer of a plurality of elongate elements 1C1, spaced apart from each other by a distance d_{1C1}, and oriented in a longitudinal direction LC1 and a layer of a plurality of elongate elements 1C2, spaced apart from each other by a distance d_{1C2}, and oriented in a longitudinal direction LC2, the directions LC1 and LC2 being intersecting, in particular forming there between an angle comprised between 85° and 95°, in particular of approximately 90°. The elongate elements 1C1 are substantially parallel to each other. Likewise, the elongate elements 1C2 are substantially parallel to each other.

Advantageously, the elongate elements (1A1, 1A2, 1B1, 1B2, 1C1, 1C2) are deposited according to the arrangement described above so as to form a base layer just after their extrusion so that they adhere to each other by means of thermofused portions acting as points of attachment by gluing.

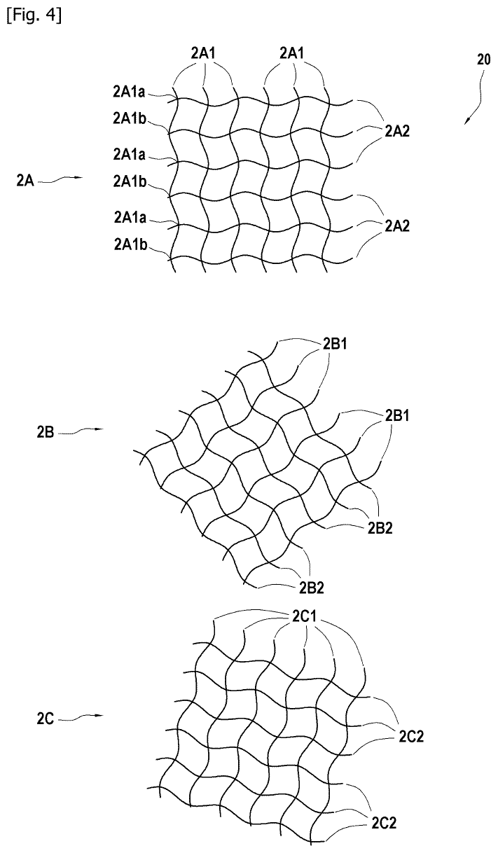

Preferably, an upper base layer (for example a layer n+1) is placed on top of a lower base layer (for example a layer n) so that the elongate elements A1 and A2 of the upper layer are substantially parallel respectively to the elongate elements A1 and A2 of the lower layer. Preferably, a base layer n+1 is similar to a base layer n, that is to say concerning the composition of the elongate elements, and their stacking together. FIG. 4 shows a second example of a base layer 20 according to the invention, which is a variant of the first example of the base layer 10 whose stack is shown in FIG. 3. The second example of a base layer 20 differs from the first example 10 in that the elongate elements (2A1, 2A2, 2B1, 2B2, 2C1, 2C2) of the base layer 20 have a sinusoidal or zigzag trajectory arranged so that they comprise alternating troughs and peaks, such as the peaks 2A1a and the troughs 2A1b shown for the elongate elements 2A1. This arrangement allows to improve the flexibility of the base layer 20. Advantageously, the elongate elements (for example 2A2 or 2B2 or 2C2) of an upper layer of a given assembly (for example 2A or 2B or 2C) are placed on top of the elongate elements (for example 2A1 or 2B1 or 2C1) of the lower layer of said given assembly (for example 2A or 2B or 2C) so that the troughs, respectively the peaks, of the elongate elements of the upper layer cooperate with the peaks, respectively the troughs, of the lower layer. This arrangement allows to improve the blocking and the hold of the elongate elements relative to each other, and allows to improve the mechanical resistance of the base structure (that is to say the hold of the structure against mechanical stresses before implantation and after implantation).

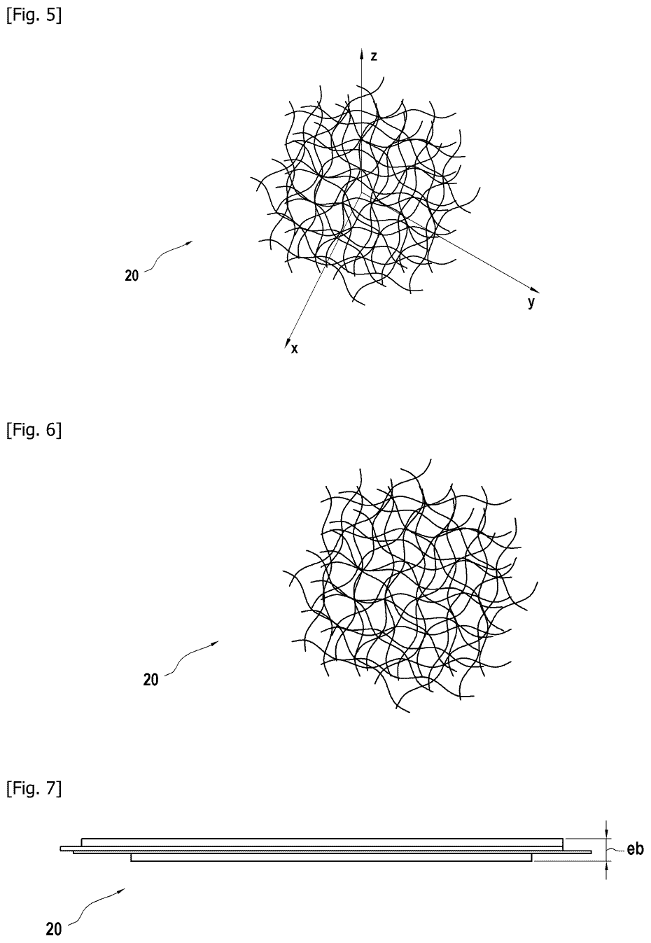

FIG. 5 shows an isometric view of the unit base layer 20 in which the three directions of space (X, Y, Z) are shown with equal importance.

FIG. 6 schematically shows a front view of the unitary base layer 20.

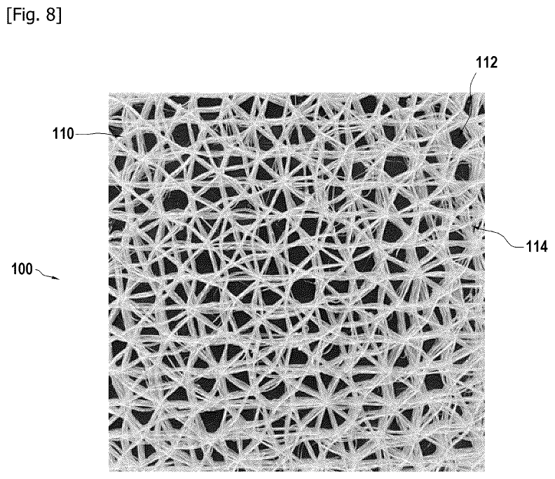

FIG. 7 shows a side view of the base layer 20 having a thickness eb of the order of 1.2 mm. FIG. 8 is a photograph showing a first example of an implantable device 100 according to the invention comprising a base structure 110 comprising a plurality of base layers placed one on top of the other until the desired final thickness is obtained.

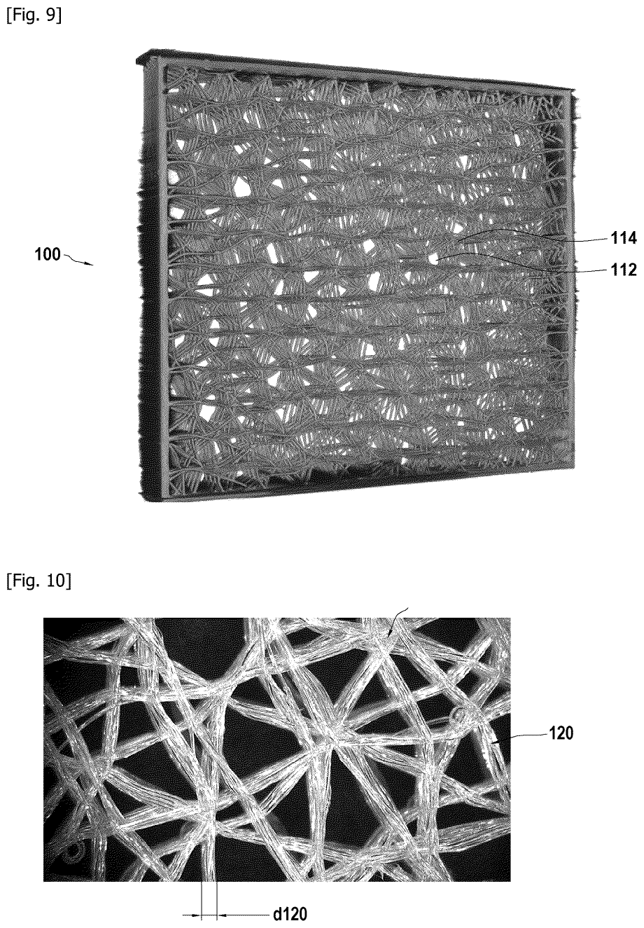

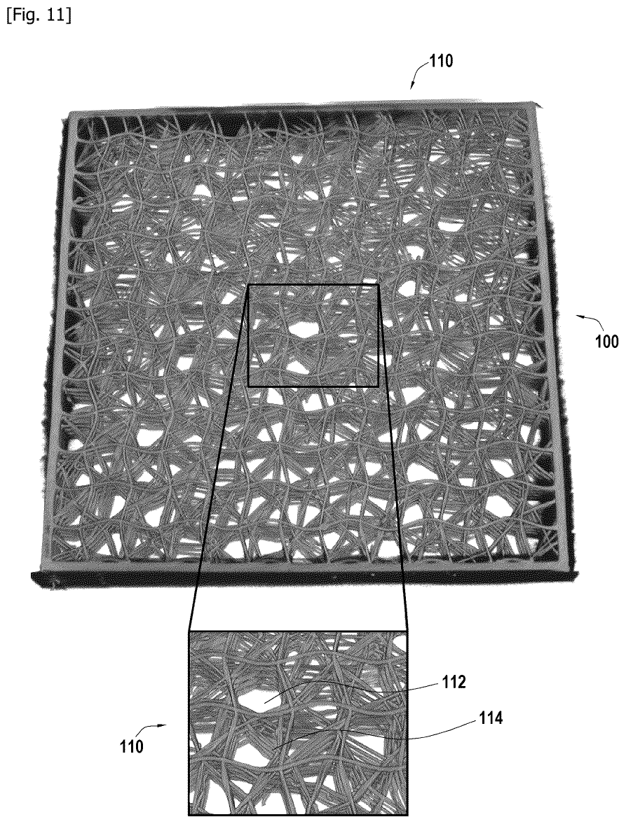

FIGS. 9 and 11 are also photographs obtained by tomography of the implantable device 100. When taking tomographic photographs, radiation (X-rays) is transmitted along a beam through the implantable device 100 placed between the beam source and a detector. The distribution of matter within the object is described by ray attenuation and spatial variables. The resolution used for analyzing the implantable device 100 is 33 microns.

In this specific example, the base structure 110 has a thickness measured between its first and second opposite faces of the order of 5 mm. The base structure 110 comprises 3 base layers 20 stacked without rotation relative to each other. The elongate elements of the base structure 110 are extruded from a copolymer of ε-caprolactone and lactic acid of L form, the mass fraction in repeating units based on ε-caprolactone is approximately 30% and the mass fraction in repeating units of lactic acid is approximately 70%. The porosity of the base structure 110 is approximately 94.5%. The density is approximately 0.04 g/cm³.

The base layers may, for example, be base layers 10 or 20. The final thickness of the base structure 110 may be adjusted by adding an assembly A, or an additional assembly A and assembly B.

As seen in FIGS. 8-11, the base structure comprises numerous open and connected pores extending between its first and second opposite faces, such as pores 112 and 114.

As can be seen in FIG. 10, the base structure 110 comprises portions of elongate elements, for example selected from the elongate elements 1A1, 1A2, 2A1, 2A2, 1B1, 1B2, 2B1, 2B2, 1C1, 1C2, 2C1 or else 2C2, having a diameter d120 or a width of the order of 0.20 mm.

FIG. 11 shows the distribution (%) of the surface size (mm²) of the pores of the base structure 110, in particular comprising stacked base layers 20.

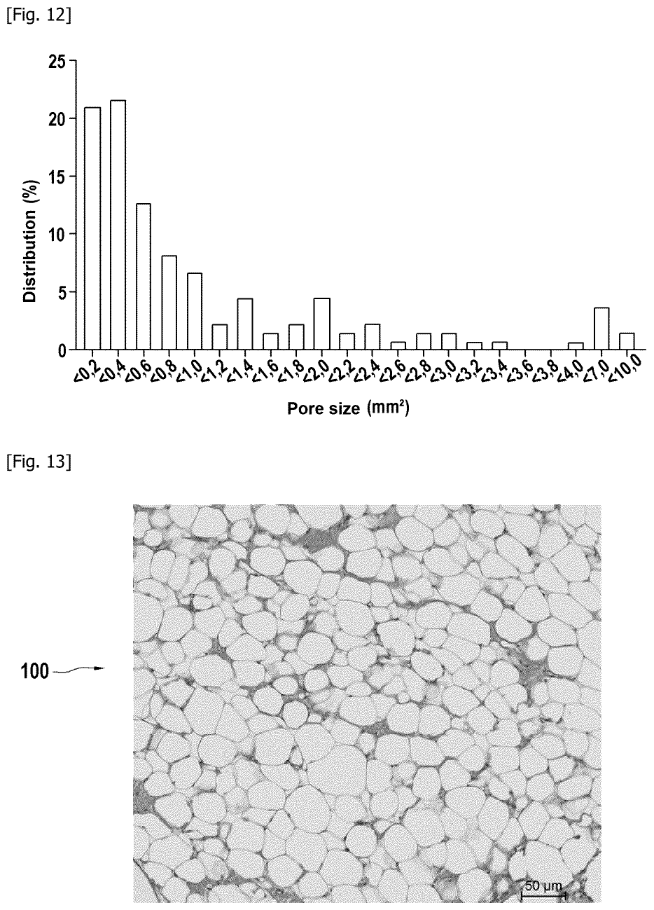

FIG. 12 shows a surface in mm² because the pore size is measured in a plane. Considering that said pore has a substantially circular size, by the formula 2*Pi*Radius², the diameter (mm) is found.

It is noted that the pores having sizes corresponding to those of the “microfat”, that is to say less than or equal to 1.25 mm, are the most significant in number in the base structure 110. Then, the pores corresponding to the millifat, that is to say between 1.25 mm and 2.5 mm, represent the second most significant category of pores, and finally the pores corresponding to the macrofat, that is to say greater than or equal to 2.5 mm, represent the least significant category of pores.

A good adipocyte density and quality corroborated by a heterogeneity in the size of the adipocytes are observed in FIG. 13 of a histological layer taken on the implantable device 100 after 2 months of implantation. The absence of space between the adipocytes and the low presence of connective tissue demonstrate the good maintenance of the adipocyte volume.

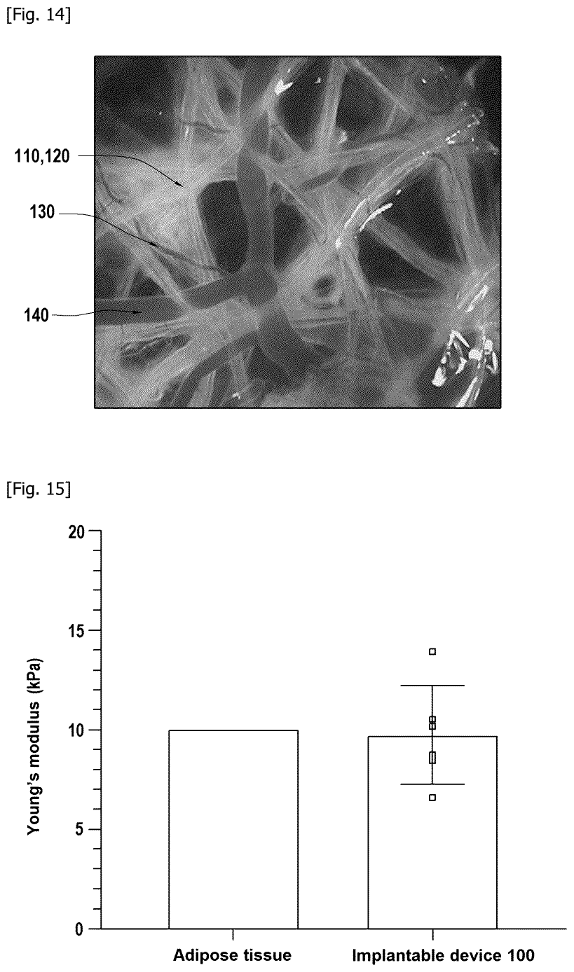

FIG. 14 shows angiogenesis of the implantable device 100 tested in FIG. 11. The base structure 110 comprises portions of elongate elements 120 and pores allowing vascularization, with the formation of small blood vessels, such as the blood vessel 130, and large-caliber blood vessels, such as the blood vessel 140. Advantageously, the distribution of the pores and their sizes allow to guide the orientation of the vessels, and thus to obtain homogeneous vascularization of the adipose tissue.

FIG. 15 shows, in a non-limiting manner, the Young's modulus obtained for the implantable device 100 comprising the base structure 110 compared to the Young's modulus of adipose tissue. It is noted that these Young's moduli are similar and on average close to 10 KPa.

It would be possible to obtain an equivalent Young's modulus for an implantable device according to the invention but with elongate elements in one or more polymer(s) different from the caprolactone and lactic acid copolymer selected for the device 100. The compression test for measuring Young's modulus was conducted according to ASTM D1621-16 “Standard Test Method for Compressive Properties of Rigid Cellular Plastics”. During the test, six standardized specimens with a diameter of 60.0 mm and a height of 26.4 mm were immersed in a fixed distilled water bath placed on the lower plate of the testing machine. The bath temperature was controlled at 37° C. The upper plate descended with a constant speed at 2.6 mm/min to compress the specimen. The cell used was 250N. The force and displacement were recorded and post-processed to obtain the elastic compression modulus.

1. A bioresorbable implantable device for the replacement and/or reconstruction and/or augmentation of soft tissue, wherein said implantable device comprises a base structure comprising at least one base layer, said base layer comprising:
an assembly A comprising a layer of a plurality of elongate elements A1 , spaced apart from each other, and oriented in a longitudinal direction LA1, and a layer of a plurality of elongate elements A2, spaced apart from each other, and oriented in a longitudinal direction LA2, the directions LA1 and LA2 being intersecting;
an assembly B comprising a layer of a plurality of elongate elements B1 , spaced apart from each other, and oriented in a longitudinal direction LB1 and a layer of a plurality of elongate elements B2, spaced apart from each other, and oriented in a longitudinal direction LB2, the directions LB1 and LB2 being intersecting;
the assembly B is placed at least partially on top of the assembly A;
the direction LA1 intersects the direction LB1;
and wherein the base structure comprises a plurality of base layers placed at least partially one on top of the other.

2. The implantable device according to claim 1, wherein the base layer (10, 20) comprises an assembly C comprising a layer of a plurality of elongate elements C1, spaced apart from each other, and oriented in a longitudinal direction LC1 and a layer of a plurality of elongate elements C2, spaced apart from each other, and oriented in a longitudinal direction LC2, the directions LC1 and LC2 being intersecting, the assembly C is placed at least partially on top of the assembly B.

3. The implantable device according to claim 1, wherein the direction LA1 intersects the direction LB1 and forms at their intersection an angle λ1 greater than 10° and less than or equal to 60°.

4. The implantable device according to claim 2, wherein the direction LA1 intersects the direction LC1 and forms at their intersection an angle λ3 greater than λ1 by at least 10°.

5. The implantable device according to claim 1, wherein the angle formed between the direction LA1 and the direction LA2 is greater than or equal to 70° and less than or equal to 110°.

6. The implantable device according to claim 1, wherein an elongate element A1 is secured to several elongate elements A2 by means of solidified thermofused portions originating from said elongate element A1 and/or the elongate elements A2.

7. The implantable device according to claim 1, wherein at least one elongate element A1 comprises at least one portion a11 of its length in a zigzag or sinusoidal shape.

8. The implantable device according to claim 7, wherein at least one elongate element A2 comprises at least one portion a22 of its length in a zigzag or sinusoidal shape, said portion a22 comprising troughs spaced apart from each other, and wherein the portion a11 of said elongate element A1 is placed on top of the portion a22 of said elongate element A2 passing through a trough of said portion a22 in a zigzag or sinusoidal shape.

9. The implantable device according to claim 1, wherein the base structure has a density greater than or equal to 0.01 g/cm³ and less than or equal to 1 g/cm³.

10. The implantable device according to claim 1, wherein at least a portion of the elongate elements selected from the elongate elements A1 , A2 , B1 , and B2 , are portions of monofilament yarns having a diameter or a width less than or equal to 0.60 mm.

11. The implantable device according to claim 1, wherein at least a portion of the elongate elements selected from the elongate elements A1, A2, B1, and B2, comprises at least one bioresorbable copolymer comprising repeating units of ε-caprolactone and repeating units of lactide of L or D or L, D form.

12. The implantable device according to claim 1, wherein the base structure has a porosity greater than or equal to 90%.

13. The implantable device according to claim 1, wherein at least 40% in number of the pores of the base structure have a size greater than or equal to 0.50 mm.

14. A method for manufacturing an implantable device according to claim 1, wherein it comprises the steps:
a step of manufacturing a base layer (i), said step comprising:
a step (i1) of manufacturing an assembly A comprising the deposition of a plurality of elongate elements A1, spaced apart from each other, and oriented in a longitudinal direction LA1, so as to form a layer, then the deposition of a plurality of elongate elements A2, spaced apart from each other, and oriented in a longitudinal direction LA2, so as to form a layer arranged above the previous layer comprising the elongate elements A1, the directions LA1 and LA2 being intersecting; and
a step (i2) of manufacturing an assembly B comprising the deposition of a plurality of elongate elements B1, spaced apart from each other, and oriented in a longitudinal direction LB1, so as to form a layer, then the deposition of a plurality of elongate elements B2, spaced apart from each other, and oriented in a longitudinal direction LB2, so as to form a layer arranged above the previous layer comprising the elongate elements B1, the directions LB1 and LB2 being intersecting;
the assembly B is placed at least partially on top of the assembly A;
the direction LA1 intersects the direction LB1;
and wherein the implantable device comprises a base structure comprising a plurality of base layers placed at least partially one on top of the other.

15. A method for ex-vivo manufacturing of an implantable device for the replacement and/or reconstruction and/or augmentation of a volume of soft tissue comprising:
the provision of an implantable device according to claim 1 comprising a base structure comprising a determined porous volume and
the arrangement, at least in part in said porous volume of the base structure of cells selected from adipocytes, cells capable of differentiating into adipocytes and mixtures of these two types of cells.

16. The implantable device according to claim 10, wherein the portions of monofilament yarns have a diameter or a width less than or equal to 0.40 mm.

17. The implantable device according to claim 2, wherein at least a portion of the elongate elements selected from the elongate elements C1 and C2 are portions of monofilament yarns having a diameter or a width less than or equal to 0.60 mm.

18. The implantable device according to claim 17, wherein the portions of monofilament yarns have a diameter or a width less than or equal to 0.40 mm.

19. The implantable device according to claim 2, wherein at least a portion of the elongate elements selected from the elongate elements C1 and C2, comprises at least one bioresorbable copolymer comprising repeating units of ε-caprolactone and repeating units of lactide of L or D or L, D form.

20. The method according to claim 15, wherein the cells are originating from the treated subject.
